(19) United States
(12) Patent Application Publication
(10) Pub. No.: US 2014/0219926 A1
(43) Pub. Date: Aug. 7, 2014

(54) Biodegradable Iron Oxide Nanoparticle Gel for Tumor Bed Therapy

(71) Applicant: The Trustees of Dartmouth College, Hanover, NH (US)

(72) Inventors: Benjamin P. Cunkelman, Acton, MA (US); Jennifer A. Tate, Hopkinton, MA (US); Alicia A. Petryk, Hanover, NH (US); P. Jack Hoopes, Hanover, NH (US); Robert J. Collier, Hanover, NH (US); Sara G. Thappa, Rockford, IL (US)

(73) Assignee: The Trustees Of Dartmouth College, Hanover, NH (US)

(21) Appl. No.: 14/171,567

(22) Filed: Feb. 3, 2014

Related U.S. Application Data
(60) Provisional application No. 61/759,852, filed on Feb. 1, 2013.

(51) Int. Cl. A61K 9/51; A61K 47/36; A61K 41/00; A61K 9/14; A61K 49/08; A61K 33/26; A61K 47/38

(52) U.S. Cl. CPC A61K 9/5161; A61K 33/26; A61K 47/36; A61K 47/38; A61K 9/14; A61K 49/08; A61K 41/00
USPC 424/9.322; 424/646; 424/493; 424/491; 424/178.1; 424/484; 607/103

(57) ABSTRACT

A biodegradable iron oxide nanoparticle gel for hyperthermia treatment of cancer includes a polysaccharide-based carrier matrix and starch-coated iron oxide nanoparticles. The gel has sufficient deformability to integrate into a diffuse tumor site, adheres to tissue and has sufficient mechanical properties to remain in place during hyperthermia treatment. The gel releases iron oxide nanoparticles for uptake by cancerous cells at the tumor margin.

### RELATED APPLICATIONS

This application claims priority of U.S. Provisional Patent Application No. 61/759,852, filed 1 Feb. 2013.

### U.S. GOVERNMENT RIGHTS

This invention was made with government support under NCI 454CA 15 1662-01 awarded by the National Cancer Institute. The government has certain rights in the invention.

### BACKGROUND

The use of magnetic nanoparticles for targeted hyperthermia cancer therapy via alternating magnetic field (AMF) activation has shown great promise as a treatment technique, in initial testing. Specifically, iron oxide nanoparticles (IONPs) have been used extensively in in-vitro and in-vivo experiments with mice, and have been shown to be both safe and effective.

IONPs can be delivered to a tumor through direct injection. When coated with a biocompatible surfactant, such as Dextran or PEG, IONPs are taken up by the surrounding cancer cells, and aggregate within the cells. Once aggregated, IONPs are introduced into an alternating magnetic field between 30 and 300 kHz. This field puts IONPs in a hysteresis loop of alternating magnetism, which causes localized hyperthermia, thus damaging and killing cells containing the nanoparticles. While the mechanism is still debated as to whether the IONPs are damaging and killing cells through heat or through mechanical vibrations, the results are clear: Injecting IONPs is an effective localized approach to treatment of cancer cells, maximizing the therapeutic ratio.

One of the current limitations of nanoparticle technology, however, is this delivery method of direct injection of IONPs into cancer cells—typically a tumor. While this has been shown to be effective in mouse tumors, it presents problems in real-world applications. In particular, it is difficult to deliver IONPs into a diffuse tumor or into a tumor that is not located on an accessible surface. It is also difficult to achieve a high therapeutic ratio in non-isolated tumors.

Moreover, whether IONPs or more conventional surgical or radiation methods are used to treat a tumor, there are often remnant cancer cells left on the margins of a tumor bed upon tumor resection. These cells present an entirely new problem, as they must be treated using a more diffuse and harmful method such as chemotherapy. These remaining cancer cells are also more difficult to treat due to their physical dispersion along the tumor bed.

### SUMMARY

Once a tumor is surgically removed, cancerous cells may remain in the tumor bed. For instance, a study of head and neck cancer conducted by Brennan et al. and reported in “Molecular Assessment of Histopathological Staging in Squamous-Cell Carcinoma of the Head and Neck”, the New England Journal of Medicine, Feb. 16, 1995, found that tumor cells were present in 50% of surgical resection margins that appeared to be microscopically free of disease. “The Hydra Phenomenon of Cancer: Why Tumors Recur Locally after Microscopically Complete Resection”, Cancer Research by Hockel and Dornhofer, likewise reports a 50% recurrence of solid malignant lung cancer after surgical resection with microscopically clear margins.

The present invention provides an improved and targeted delivery vehicle for IONPs that is suitable for treating both recurrent cancer cells on a tumor bed and cancer cells that have metastasized (i.e., to the lymph nodes), and which increases the application, efficacy and therapeutic ratio of IONPs. The therapeutic gels disclosed herein advance the art of hyperthermia cancer therapy by providing a deformable gel that adheres directly to tissue and has sufficient mechanical properties to remain in place during hyperthermia treatment, while draining from the tumor bed to the lymph nodes after therapy. As the IONPs follow the route of cancer metastasis, the lymph nodes may be easily treated by hyperthermia therapy after primary treatment of the tumor bed. The present invention thereby enhances and improves treatment of cancer, while also contributing to a disclosed diagnostic method of locating and tracking the path of cancer through the body.

In one embodiment, a biodegradable iron oxide nanoparticle gel includes a polysaccharide and iron oxide nanoparticles. The gel has sufficient deformability to integrate into a diffuse tumor site. The gel adheres to tissue and has sufficient mechanical properties to remain in place. The gel releases the iron oxide nanoparticles for uptake by cancerous cells at the tumor site, and maintains its mechanical properties at temperatures ranging from room temperature to a hyperthermia range of 43-37° C.

In one embodiment, a method of targeted hyperthermia treatment following resection includes applying a biodegradable iron oxide nanoparticle gel to a tumor bed following resection, allowing the gel to remain on the tumor bed for an elution period and heating the nanoparticle gel using an external AC magnetic field. The gel is allowed to drain from the tumor bed for a drainage period, and the external AC magnetic field is applied proximate a lymph node or nodes within the lymphatic drainage of the tumor bed, to heat nanoparticles metastasized from the tumor bed to the lymph node or nodes and ablate cancer cells within the lymph node or nodes.

In one embodiment, a diagnostic method for locating and mapping cancerous cells within a patient's body includes applying an iron oxide nanoparticle gel to a tumor bed, following resection, allowing the gel to sit for an elution period, and allowing the gel to drain for a drainage period. One or more imaging studies is performed to image iron oxide nanoparticles eluted from the gel and taken up by cancerous cells within the body. Imaging is repeated until mapping of cancerous cells within the body is complete.

In one embodiment, a biodegradable iron oxide nanoparticle gel includes a carrier matrix including fibrin glue and biocompatibly-coated iron oxide nanoparticles. The gel has sufficient deformability to integrate into a diffuse tumor site. The gel adheres to tissue and has sufficient mechanical properties to remain in place. The gel releases the iron oxide nanoparticles for uptake by cancerous cells at the tumor site, and maintains its mechanical properties at temperatures ranging from room temperature to a hyperthermia range of 43-37° C.

In one embodiment, a biodegradable iron oxide nanoparticle gel includes a carrier matrix including collagen and biocompatibly-coated iron oxide nanoparticles. The gel has sufficient deformability to integrate into a diffuse tumor site. The gel adheres to tissue and has sufficient mechanical properties to remain in place. The gel releases the iron oxide nanoparticles for uptake by cancerous cells at the tumor site, and maintains its mechanical properties at temperatures ranging from room temperature to a hyperthermia range of 43-37° C.

### BRIEF DESCRIPTION OF THE DRAWINGS

FIG. 1 illustrates the chemical structure of pullulan.

FIGS. 2A-2C are photographs showing three biocompatible iron oxide nanoparticle gels for tumor bed therapy.

FIG. 3A-3C are photographs showing the gels of FIG. 2A-2C, respectively, following addition of water and incubation.

FIG. 4 is a flow chart illustrating a method 200 of targeted hyperthermia treatment following resection of a tumor.

FIG. 5 is a flow chart illustrating a diagnostic and tracking method 300 for mapping cancer cells within a patient's body.

FIGS. 6A, 6B, and 6C show histological sections of lymph nodes from canine patients in dog trials of the disclosed IONP gels, demonstrating migration of IONPs from a site of gel deposit to the lymph nodes.

FIG. 7A is a photograph showing canine oral melanoma surrounding an upper canine tooth of a canine patient in a dog trial described herein.

FIG. 7B shows iron oxide nanoparticle gel placed with the tumor bed following removal of the melanoma shown in FIG. 7A.

FIG. 7C shows normal periodontal tissue six months following hyperthermia treatment with the iron oxide nanoparticle gel of FIG. 7B.

FIG. 8 illustrates the chemical structure of fibrin.

FIG. 9 illustrates the chemical structure of fibrinogen.

FIG. 10 illustrates the chemical structure of thrombin.

### DETAILED DESCRIPTION

Treatment of the post-operative surgical bed is appealing because the majority of cancer recurrence following tumor resection occurs at the tumor margin. A gel formulation is also appealing, as it allows the possibility of spreading or injecting over the surface of the surgical bed. In order to perform effectively for delivery of IONPs into cancer cells at the surgical bed, gels were formulated to meet specifications of biocompatibility, a balance between localized toxicity at the surgical site and toxicity to the rest of the body, a balance between short and long term toxicity, deformability, temperature consistency, high IONP concentration capacity, stability and IONP release capability.

In one aspect, a polysaccharide-based gel infused with starch-coated magnetic iron oxide nanoparticles is applied to the post-operative wound site. Polysaccharides used in the gel include, but are not limited to pullulan, cellulose, amylose, amylopectin, agarose, glycogen, chitin, callose, laminarin, chrysolaminarin, xylan, a hemicellulose such as arabinoxylan, mannan, fucoidan and galactomannan, alone, in combination with one another. The polysaccharide may be pre-retrograded (pre-heated to retrograde the starch and enhance viscosity of the gel) prior to its inclusion in the gel, or the fully prepared gel may be heated to retrograde the polysaccharide post-mixing.

A protein or protein precursor (such as fibrin or collagen, or fibrinogen) may also be added to the gel to enhance viscosity and/or promote tissue healing at the surgical site. Optionally, the polysaccharide may include the protein/precursor without the polysaccharide. Still optionally, a polysaccharide derivative, such as carboxymethylcellulose or agar, may provide the gel matrix, alone or in combination with a protein/protein precursor, a polysaccharide, water or saline.

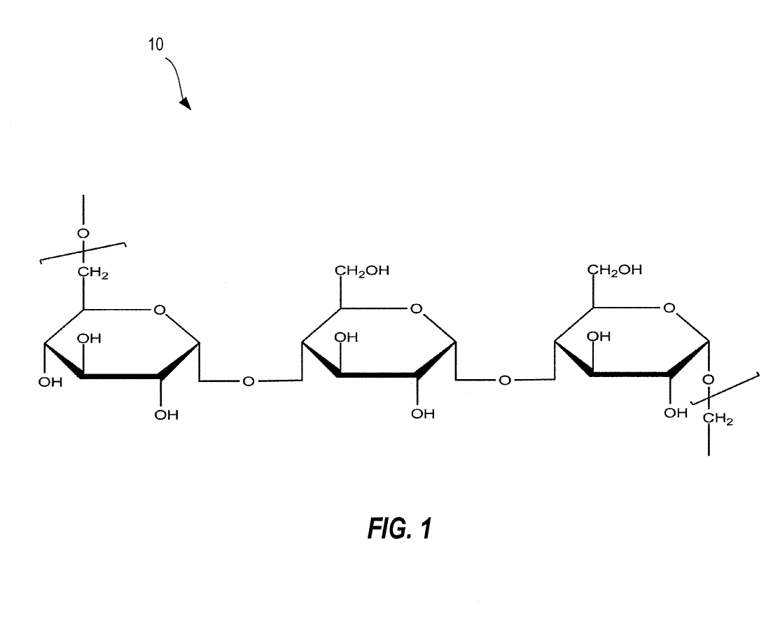

FIG. 1 illustrates the chemical structure 10 of pullulan, or α-1,4-; α-1,6-glucan, which is a water soluble, film-forming starch polymer. Pullulan is compatible with starch-coated IONPs, biocompatible, and has proven to degrade at the post-operative wound site to deliver an IONP payload, which is taken up by cancer cells remaining at the surgical margins.

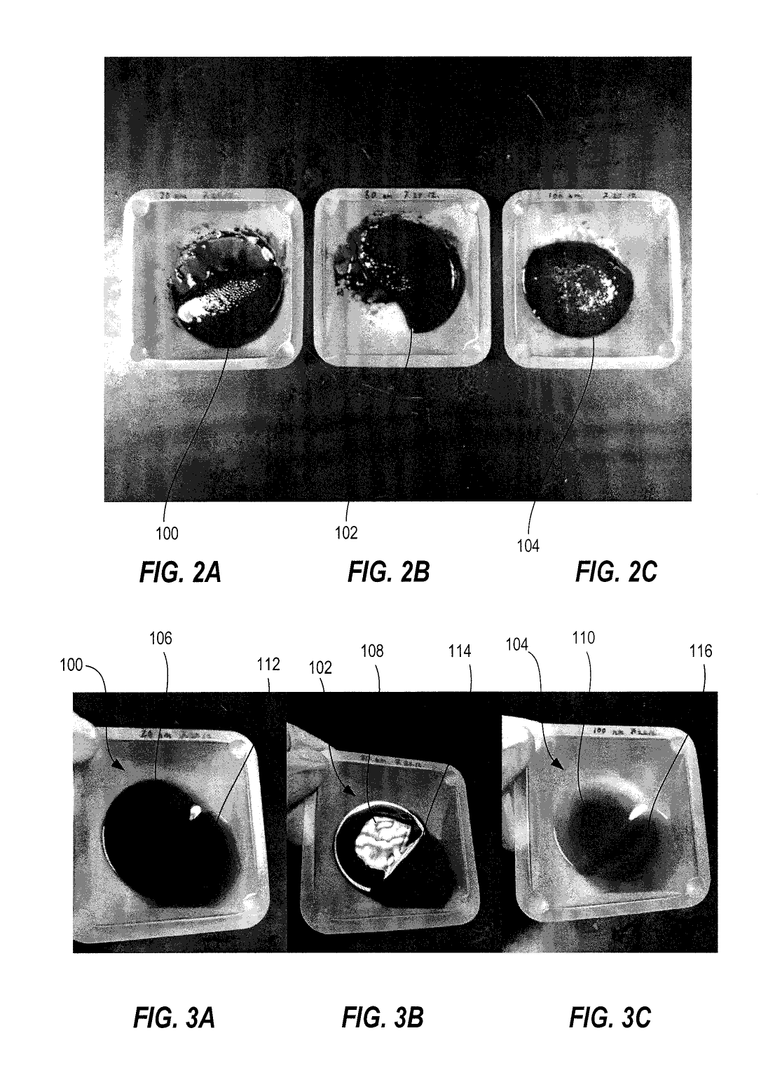

FIGS. 2A-2C show three pullulan-based gels 100, 102 and 104, respectively. Gel 100 (FIG. 2A) is a mixture of pullulan, phosphate buffered saline (“PBS”) and 20 mg nanomag-D spioplain IONPs at a concentration of 108 mg/mL. Gel 102 (FIG. 2B) is a mixture of pullulan, PBS and 80 nm BNF Dextran IONPs at a concentration of 38 mg/mL. Gel 104 (FIG. 2C) is a mixture of pullulan, PBS and 100 nm nanomag-D spioplain at a concentration of 10 mg/mL. Pullulan and PBS may be combined at a ratio of approximately 3 g pullulan to approximately 7 mL PBS, and incubated for a period of time prior to addition of the IONPs; for example, for 24 hours at 37° C. However, it will be appreciated that pullulan and PBS may be otherwise combined, and that IONPs may be added to the mixture prior to incubation.

Gels 100-104 are thick, stable and highly viscous hydrogels that are biodegradable and capable of releasing IONPs at the site of application. After addition of water and incubation, gels 100-104 showed significant degradation after 1 hour. FIGS. 3A-3C show gels 100, 102 and 104 separated into remaining gel portions 106, 108 and 110 and respective IONP liquid portions 112, 114 and 116, after water was added to gels 100-104 and the water/gel combinations incubated. Note dark liquid 112 separated from remaining gel portion 106 of gel 100 in FIG. 3A, dark liquid 114 separated from remaining gel portion 108 of gel 102 in FIG. 3B, and dark liquid 116 separated from remaining gel portion 110 of gel 104, in FIG. 3C. Liquids 112, 114 and 116 were carefully examined and shown to carry IONPs in solution. Gels 100-104 degrade rapidly and both carry and elute high concentrations of IONPs. Gels 100-104 remain equally stable at room temperature and at 37° C., and their mechanical properties did not change measurably. See Experimental Results, below, for additional detail.

The biodegradable iron oxide nanoparticle gels disclosed herein show great promise in treating cancer cells surrounding a tumor bed via hyperthermia therapy. In addition to their elution capabilities, the gels themselves are still susceptible to bulk heating, and could be topical treatments for cancer cells on the surface of a wound-bed after tumor resection.

In order to maximize therapeutic benefits of the disclosed gels, the IONPs may be coated with a biocompatible antibody target, to encourage uptake and aggregation of the IONPs within cancerous cells at or proximate the treatment site. The disclosed gels may also be coated via laparoscopic techniques onto tumors and incubated for a period of time to secrete IONPs into the cancer cells. Targeted uptake using a biocompatible gel represents an advance over conventional mass-diffusion of IONPs within the bloodstream, in which IONPs may aggregate within healthy cells.

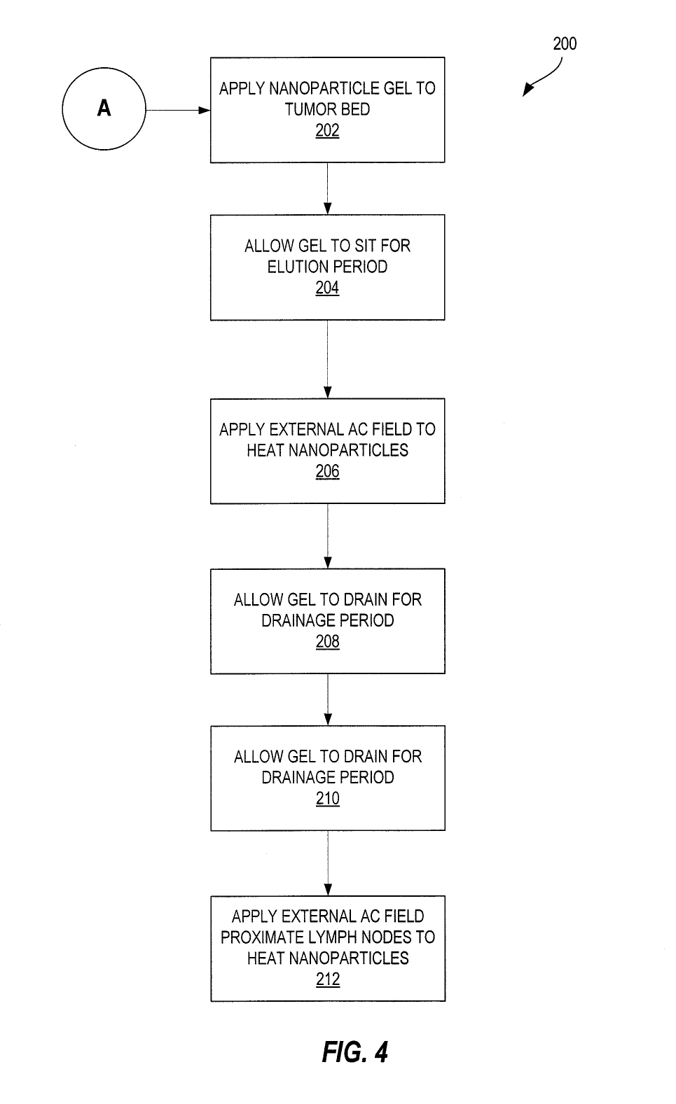

FIG. 4 illustrates a method 200 of targeted hyperthermia treatment following resection of a tumor. In step 202, an IONP gel is applied to a tumor bed following surgical resection. In one example of step 202, gel 100, 102 or 104 is injected, laparoscopically coated or otherwise applied to a tumor bed/tumor margins following removal of the tumor. In step 204, the gel is allowed to sit for an elution period. In one example of step 204, the gel is allowed to sit for 30 minutes to 4 hours. It will be appreciated that the elution time is selected based upon elution of the gel formulation, and may therefore vary depending upon elution capabilities of the gel. In step 206, a coil is positioned external to the patient's body and proximate the resection site, and an AC field is applied to excite and heat the IONPs at the tumor bed. As the IONPs bind with cancer cells, heat-induced damage to healthy tissue is minimized, while maximizing hyperthermia of remaining cancer cells.

Step 208 represents a drainage period. Following step 206, the gel is allowed to elude and drain from the tumor bed, or more specifically, IONPs are allowed to drain from the gel, for a predetermined period. In one example; after a drainage period of 1-4 hours (step 210), the coil is repositioned proximate to the tumors primary draining lymph nodes, i.e., lymphatic drainage of the tumor bed. Then an external AC field is applied to the lymph node/NPs to generate cytotoxic heat in the cancer cells residing in the lymph nodes (step 212). As the IONPs bind to tumor tissue or macrophages, hyperthermia of healthy tissue within the lymph nodes is minimized, while cancer cells may be destroyed. Method 200 therefore provides for treatment of tumor cells remaining at tumor margins—even margins that are microscopically free of disease, while also treating the metastatic path of the tumor. It will be appreciated that step 212 may be repeated at various sites in order to further treat the lymphatic drainage associated with a tumor bed. In another example of steps 210-212, depending upon spread and concentration of cancerous cells within the body, the draining lymph node or nodes could be effectively treated with the alternating magnetic field from 4-21 hours after primary treatment of the tumor bed (step 206).

In one example of treatment, gel 100, 102 and/or 104 is applied to a resection site post-lumpectomy or post-mastectomy. After the elution period (step 204), an external AC field is applied to heat nanoparticles and associated cancer cells at the resection site. Following a second waiting period (the drainage period, step 208), regional lymph nodes in the chest wall (for example) are treated with the AC field to induce hyperthermia of cancer cells that may have metastasized.

In order for IONPs to be a maximally effective therapy, they must be specifically targeted into tumors. Currently, research is being done to coat IONPs with a biocompatible antibody target. This would be recognized by cancer cells, which would then take up the IONPs, causing them to aggregate within the cancerous cells themselves. The current proposed delivery method is through mass-diffusion within the blood stream. Though healthy cells will hopefully not take up the IONPs, they may still aggregate within some tissues, such as the liver or spleen. While such diffusion may be the only solution for treating metastases, it is a poorly targeted method for addressing discrete tumors.

A biocompatible gel would be an ideal solution. Ultimately, a gel that could be coated via laparoscopic techniques onto tumors and incubated for a period of time to secrete IONPs into the cancer cells would maximize the therapeutic ratio for this treatment. The mechanical properties of gels in general would be ideal for concentrating the IONPs into the tumor and reducing the amount of IONPs taken into healthy tissue or into other organs.

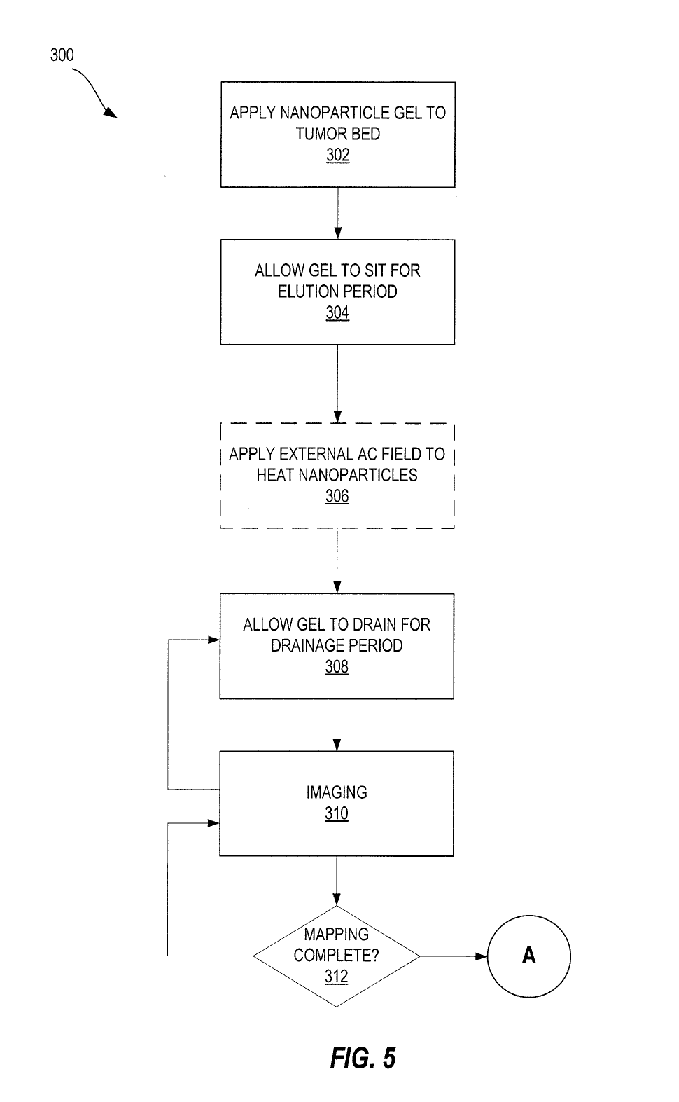

FIG. 5 illustrates a diagnostic (and optionally, treatment) method 300 of detecting and mapping cancerous cells within a patient's body. IONP gel is applied to a tumor bed following resection, in step 302, and allowed to sit for an elution period, in step 304. An external current may be applied to generate an alternating magnetic field to heat the nanoparticles, in optional step 306.

The gel is allowed to drain for a drainage period, in step 308. Imaging (Step 310) occurs periodically during the drainage period, which may be up to 24 hours. In one aspect, SWIFT MRI is used in imaging step 310, in order to visualize the IONPs within bone and other tissue.

Step 312 is a decision. If mapping is complete, method 400 may finish or hyperthermia treatment method 200 may commence.

In one example of method 300, a polysaccharide-based IONP gel is coated or injected (e.g., laparoscopically) over a tumor bed and allowed to sit for 30 minutes to four hours (Steps 302 and 304). Hyperthermia treatment may be performed after the elution period, to treat and destroy cancerous cells remaining at the tumor margins (optional step 306). During a drainage period of up to 24 hours, SWIFT MRI may be periodically performed to image accumulation of the IONPs within cancer cells in bone, lymph node or other tissue. A time of gel placement may be recorded, and MRI images may be time-stamped so that elapsed time corresponding to optimal concentration of IONPs at various sites in the body may be determined.

Once mapping of cancer within the body is complete, treatment method 200 may commence or be scheduled for a later date. Treatment at various sites of metastasis (i.e., by method 200) may be timed to correspond with optimal concentrations of IONPs at individual metastatic sites, as determined by comparing gel placement time with optimal concentrations seen in time-stamped MRI images.

### Experimental Results

### Gel Development

NaHCO₃, H2O, gelatin, glycerin, NaCl, guar gum, flour, xanthan gum, methyl cellulose and pullulan (a polysaccharide powder) were tested in various combinations for their efficacy as an IONP gel. Thickening ½ mL of IONPs with 2 g of a 2 tablespoon xantham gum and 1 tablespoon flour mixture yielded a gel that was not sufficiently viscous. Likewise, adding ½ mL of IONPs to a mixture of ¾ cup NaHCO₃, ¼ cup NaCl, and ¼ oz. gelatin proved to have too low a viscosity. Adding ½ mL IONPs to ¾ cup NaHCO₃, ¼ cup NaCl, H₂O, and 12 teaspoons of glycerin produced the same undesirable result. Mixtures of guar gum and flour as well as mixtures of xanthan gum and flour, more explicitly 2 tablespoons of guar gum and 1 tablespoon of flour with ½ mL of IONPs and 2 tablespoons of xanthan gum and 1 tablespoon of flour with ½ mL of IONPs, were more viscous but tended to break apart into separate chunks. Biocompatibility of xanthan gum and guar gum was also questionable. However, all of these compounds exhibited shelf stability.

Polysaccharide formulations were also investigated. Pullulan emerged as an ideal polysaccharide, given its film-forming capabilities and its compatibility with starch-coated IONPs. In a series of deformability tests, 1 g of pullulan mixed with 9 mL PBS, after incubation for 24 hours at 37 C, produced a gel with a viscosity that was too low. However, a 3 g pullulan and 7 mL PBS gel, after undergoing the same incubation process, had an ideal viscosity for an IONP delivery vehicle.

Next, pullulan's efficacy in forming a biodegradable gel was tested with three different types of IONPs: (1) 20 nm nanomag-D spioplain IONPs at a concentration of 108 mg/mL (gel 100, FIG. 2A); (2) 80 nm BNF Dextran IONPs at a concentration of 38 mg/mL (gel 102, FIG. 2B); and (3) 100 nm nanomag-D spioplain at a concentration of 10 mg/mL (gel 104, FIG. 2C). Regardless of IONP type, pullulan ((C6H12O5)n) formed a thick, high viscosity hydrogel with the IONPs, and these gels proved stable.

With these specifications of deformability, compatibility with the IONPs, and stability met, biodegradability and IONP release capacity of the pullulan gel were tested. In order to best understand the biodegradability of the gel and its elution properties, 10 mLs of H₂O were added to each of the three IONP gel formulations described above. These samples were stored at 37° C. and incubated for 1 hour. After 1 hour incubation, the gels were first examined by inspection to determine efficacy of biodegradability. All three gel formulations showed significant degradation after 1 hour.

The IONP liquid from each gel formulation (liquid 112 for gel 100; liquid 114 for gel 102; liquid 116 for gel 104, see FIGS. 3A-3C) was then removed from the gel via pipette and put in separate 15 mL conical tubes for elution testing. The concentrations of IONPs that had eluded into the sample liquids were tested. Absorbance spectroscopy was used to shine light through the samples and determine the concentration of nanoparticles by comparing the absorbance value of the samples to known baseline values of solutions with different concentrations of nanoparticles. The elution data from absorbance spectroscopy is being processed, Greater concentrations appear to result in longer elution time and greater concentration of iron in the tumor bed.

10 mL of H₂O was then added to gel formulations 106, 108 and 110 that remained after the respective IONP liquid 112, 114 or 116 was removed, and the water and gel combinations were incubated overnight at 37° C. After overnight incubation, the entire gel formulation poured off dark with no gel or gel film left on the bottom of the tray. This shows that the pullulan and IONP gel formulations have the ability to degrade rapidly and both carry and elute high concentrations of IONPs. Moreover, this initial testing revealed that the gels remained equally stable at both room temperature and at 37° C., and that mechanical properties of the gels did not change measurably.

The pullulan and IONP gels showed great promise in treating cancer cells surrounding a tumor bed via hyperthermia therapy. In addition to their elution capabilities, the gels are still susceptible to bulk heating, and could be a topical treatment for cancer cells on the surface of a wound-bed after tumor resection.

### Dog Trials

In a trial of hyperthermia treatment with an IONP gel, 12 dogs diagnosed with oral cancer underwent baseline studies of blood iron, metabolics, cytokines, histological diagnosis, and circulating tumor cells (CTCs). Histological diagnosis and/or tumor biopsy was performed. Excision surgery, radiation and/or Cisplatin chemotherapy were performed. A polysaccharide IONP gel having a matrix of 3 g pullulan to 7 mL PBS, mixed with IONPs, was applied to the tumor bed at a strength of about 5 mg Fe per gram of tumor, and left in place for a period of time. The dogs were then subjected to an alternating magnetic field to induce hyperthermia (for example, by placing coils beneath a table, positioning the dog with the tumor site over and proximate the coils, and activating the coils to produce the magnetic field). The above-noted baseline studies were repeated at or close to treatment time, and again at a follow-up visit.

The dogs had ameloblastoma (A), a non-metastasizing but readily reoccurring cancer that starts at the root of a tooth and invades into bone, acanthomatous ameloblastoma (AA), a rare variant of ameloblastoma, or oral melanoma (M), an aggressive, metastasizing cancer. Seven dogs are represented in Table 1, below. As shown, two dogs with ameloblastoma were cancer-free at their follow-up visits (with one dog cancer-free at 19 months out, and the other cancer-free at 25 months out). One dog with oral melanoma, which generally carries a life expectancy of about five months from diagnosis, was cancer free at five months out.

### Canine Oral Cancer Mnp-Amf Rx Trial

TABLE 1
magnetic nanoparticle gel and alternating magnetic field trial of canine patients
with Ameloblastoma (A), Acanthomatous Ameloblastoma (AA) and Melanoma (M)

| Canine Patient | Schnauzer | Labrador | Poodle | Labrador | Scottie | Pug | Lapphund |
|---|---|---|---|---|---|---|---|
| Tumor | A | A | AA | M | M | M | M |
| Type | | | | | | | |
| Tumor Size | 2 cm³ | 2 cm³ | 2 cm³ | 5 cm³ | 2 cm³ | 1 cm³ | 2 cm³ |
| Tumor | Incisor | Canine | Canine | Premolar | Molar | Premolar | Molar |
| Position | region | tooth | tooth | region - L | region - L | region - L | region - L |
| | | region - R | region - L | | | | |
| Imaging | CT/MR | CT/MR | CT | CT/MR | CT | CT | CT |
| (pre/post) | (pre/post) | (pre/post) | (pre) | (pre/post) | (pre/post) | (pre/post) | |
| Radiation | 6X6 Gy | — | — | 7x6 Gy | — | 6X6 Gy | — |
| | (36) | | | (42 Gy) | | (36 Gy) | |
| Local mNP | + | + | − | + | + | + | + |
| dose: | | | | | | | |
| 5 mg/FE/gm | | | | | | | |
| tumor | | | | | | | |
| Thermal | 2X100 | 2X100 | 100 | 2X200 | 200 | 2X100 | 2X500 |
| Dose (CEM) | CEM | CEM | CEM | CEM | CEM | CEM | CEM |
| | | | | | | | (72 hrs) |
| Surgery | + | + | + | + | + | + | + |
| Follow-up | 25 months | 19 months | 2 weeks | 10 months | 4 months | 11 months | 5 months |
| | cancer | cancer | | (euthanized | local | regional | cancer |
| | free | free | | with | recurrence | lymph | free |
| | | | | metastasis) | | node met | |
| | | | | | | local | |
| | | | | | | recurrence | |

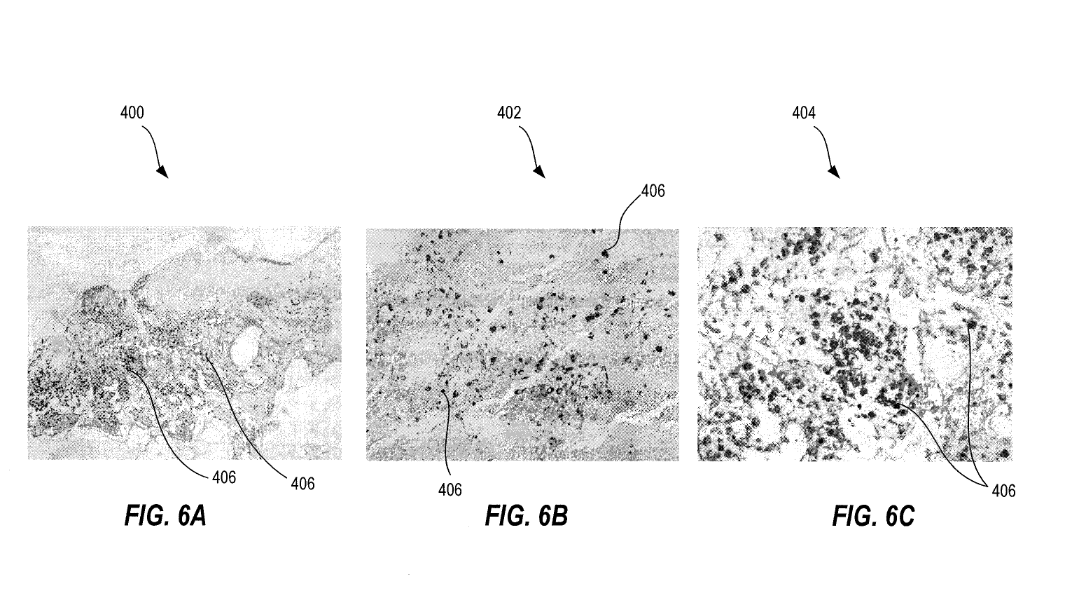

Mandibular lymph nodes removed from one dog at four hours post-treatment with IONP gel were enlarged and hardened upon examination. Histological sections 400, 402 and 404, shown in FIGS. 6A, 6B and 6C, revealed that IONPs had migrated from the oral tumor site of gel deposit to the mandibular lymph node, where they had been taken up by metastatic tumor cells and macrophages. Note the presence of stained IONPs 406 in sections 400-404 (not all IONPs are specifically labeled).

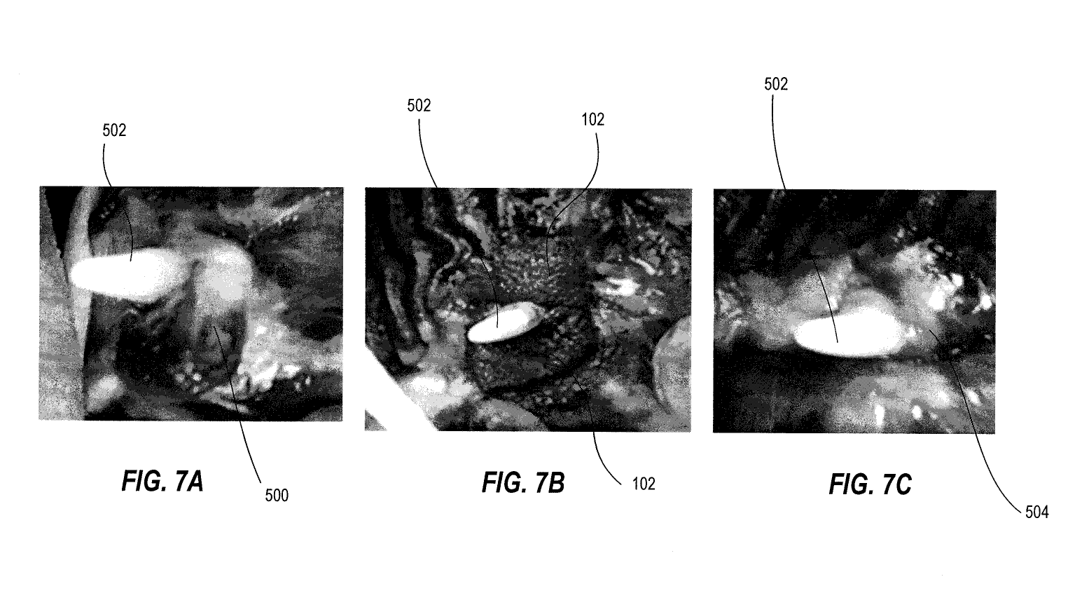

FIGS. 7A-7C together illustrate results of hyperthermia treatment of an oral melanoma 500 proximate canine tooth 502 of one dog treated in the canine trials. FIG. 7A shows melanoma 500 prior to excision and IONP treatment. FIG. 7B shows IONP gel 102 placed with the tumor bed (not visible) about tooth 502. FIG. 7C shows healthy gum tissue 504 in place of melanoma 500, six months after hyperthermia treatment with IONP gel 102.

The dog trials demonstrated that a polysaccharide IONP gel is effective in coating and delivering an IONP payload to a tumor bed, and furthermore, that IONP metastasis follows the path of cancer metastasis to the lymph nodes. IONPs were found to bind either with macrophages, which readily replaced by the body, or by cancer cells. Gels 100 and 102 appeared to be most effective in the dog trials. Although the pullulan-based IONP gels proved to be effective delivery vehicles, they melted rather quickly upon activation of the alternating magnetic field, and appeared to affect the ability of the nanoparticles to generate heat. A more stable gel may be less likely to diffuse away from the tumor bed and/or interfere with heat production, and may therefore be even better able to deliver IONPs to cancer cells at the surgical margins.

Although effective aids to hyperthermia treatment, the original pullulan formulations lost viscosity more quickly than anticipated following AMF exposure and NP tumor bed heating. In response more viscous pullulan-alternative polysaccharide mixtures are being tested. It is anticipated that for even greater efficacy, the IONP gel should retain a semi-solid consistency for at least 30 minutes following the initiation of the alternating magnetic field.

### Enhancements

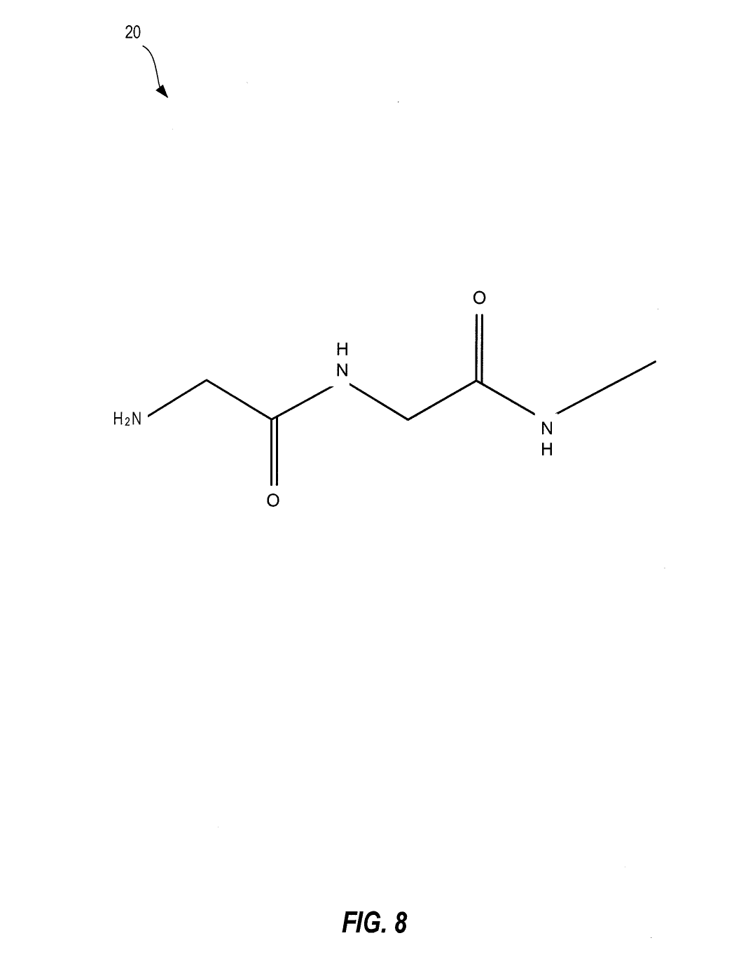

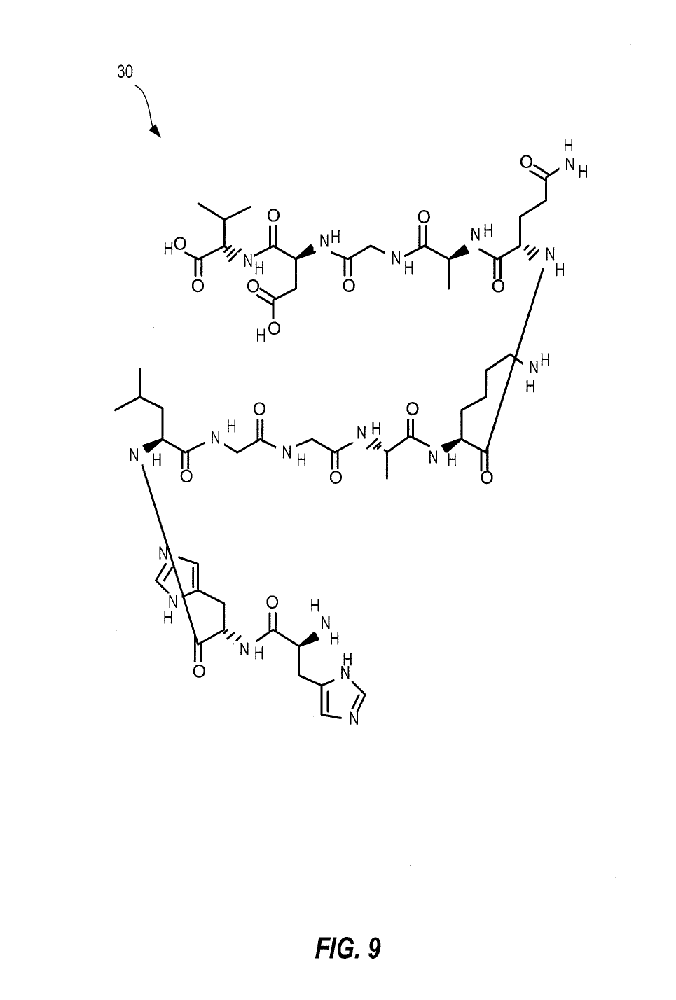

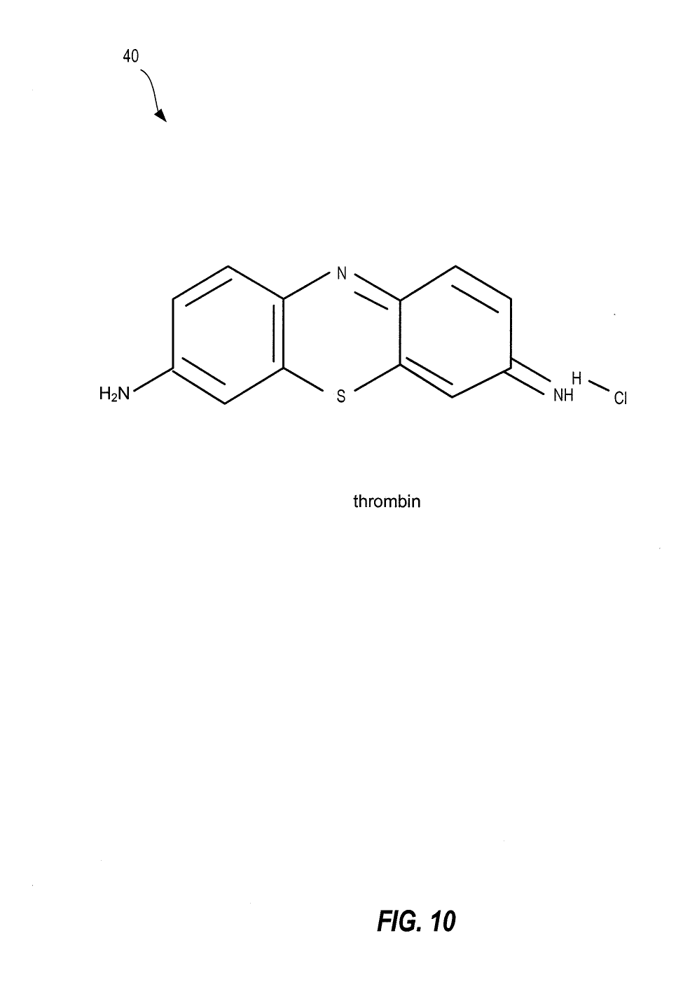

Contemplated enhancements to the IONP gel include adding fibrin or fibrinogen (FIG. 9) to the pullulan-based gel. In a fibrinogen-based gel, thrombin may be added to convert the fibrinogen to fibrin. For example, fibrin glue (a combination of fibrinogen and thrombin) may be used. FIG. 8 shows chemical structure 20 of fibrin, FIG. 9 shows the chemical structure 30 of fibrinogen, and FIG. 10 shows the chemical structure 40 of thrombin. Alternately, fibrin, fibrinogen or another protein may replace the polysaccharide in the IONP gel, or may be combined with a different polysaccharide/starch to form the IONP gel matrix. One contemplated gel includes carboxy-methylcellulose, biocompatible-coated IONPs and fibrinogen. Fibrin/fibrinogen may beneficially help keep the tissue together during treatment. Agar may also contribute to a suitable gel matrix, as may collagen. In one aspect, the IONP gel may have a collagen-thrombin matrix similar to D-Stat.

A polysaccharide/starch gel may also be heated in order to retrograde the gel to enhance its viscosity. For example, the gel may be retrograded prior to adding the IONPs. In another example, the IONP polysaccharide/starch gel may be fully mixed (including the IONPs) prior to heating. Heat may be applied to the gel prior to coating the tumor bed or, depending upon the polysaccharide used, heat generated by the IONPs during application of the magnetic field may be sufficient to heat and retrograde the gel when it is in place upon the tumor bed. Pullulan, for example, may not be useful in a retrograded polysaccharide gel, as it is known to prevent retrogradation of starch.

Polysaccharides contemplated for use in the IONP gel include, but are not limited to: pullulan, cellulose, amylose, amylopectin, agarose, glycogen, chitin, callose, laminarin, chrysolaminarin, xylan, a hemicellulose such as arabinoxylan, mannan, fucoidan and galactomannan, alone, in combination with one another and/or with a protein or protein precursor (including but not limited to fibrinogen/fibrin or collagen).

In order for IONPs to be a maximally effective therapy, they may be specifically targeted into tumors. IONPs may be coated with or otherwise incorporate a biocompatible antibody target recognizable to cancer cells, which may then take up greater amounts of IONPs, causing aggregation within the cancerous cells themselves. The currently proposed delivery method is through mass-diffusion within the blood stream. Though healthy cells would hopefully not take up the IONPs, they may still aggregate within some tissues, such as the liver or spleen. While such diffusion may be the only solution for treating some metastases, it is a poorly targeted method for addressing discrete tumors.

A biocompatible gel would be an ideal solution. Ultimately, a gel that could be coated via laparoscopic techniques onto tumors and incubated for a period of time to secrete IONPs into the cancer cells would maximize the therapeutic ratio for this treatment. The mechanical properties of gels in general would be ideal for concentrating the IONPs into the tumor and reducing the amount of IONPs taken into healthy tissue or into other organs. In addition, the demonstrated ability of the IONPs to metastasize from the gel into tumor tissue within the lymph nodes allows for secondary treatment of metastatic cancer.

In one aspect, the disclosed IONP gel enables a diagnostic method of locating cancer, and a method of treating cancer along its metastatic path. IONP gel treatment and AMF-induced hyperthermia may be combined with Sweep Imaging with Fourier Transform (SWIFT) MRI in order to visualize cancer within the body—even within hard tissue such as bone. For example, following treatment and a drainage period as explained above and with reference to FIG. 4, the SWIFT method may be used to visualize IONPs within cancer cells in the lymph nodes, in bone or in other tissue so that a customized treatment plan may be made for an individual patient. Following “plotting” of cancer cells within a patient's body, secondary IONP delivery and sequential activation of an AC coil proximate sites of cancer accumulation may be carried out in order to treat cancer along the metastatic path. Use of SWIFT MRI with the disclosed IONP gel may also aid in better understanding the metastatic path of certain cancers.

The disclosed IONP gel may be useful in treating human cancers at surgical resection margins and at sites of metastasis throughout the body.

While the present invention has been described above, it should be clear that many changes and modifications may be made to the process and product without departing from the spirit and scope of this invention.

### Combinations of Features

Features described above as well as those claimed below may be combined in various ways without departing from the scope hereof. The following examples illustrate possible, non-limiting combinations of features of the inventions described above. It should be clear that many changes and modifications may be made to the systems and methods described above without departing from the spirit and scope of this invention:

(a) A biodegradable iron oxide nanoparticle gel includes at least one polysaccharide and iron oxide nanoparticles. The gel has sufficient deformability to integrate into a diffuse tumor site. The gel adheres to tissue and has sufficient mechanical properties to remain in place. The gel releases the iron oxide nanoparticles for uptake by cancerous cells at the tumor site, and the gel maintains the mechanical properties at temperatures ranging from room temperature to a hyperthermia range of 43-37° C., for at least a therapeutic time period.

(b) In the gel denoted as (a), the polysaccharide may be pullulan, cellulose, amylose, amylopectin, agarose, glycogen, chitin, callose, laminarin, chrysolaminarin, xylan, a hemicellulose such as arabinoxylan, mannan, fucoidan, galactomannan and derivatives thereof, alone or in combination.

(c) In the gel donated as (a) or (b), the gel may include a mixture of the at least one polysaccharide, phosphate buffered saline (“PBS”) and starch-coated iron oxide nanoparticles (“IONPs”).

(d) In the gel donated as (a) or (b), the iron oxide nanoparticles may be starch-coated iron oxide nanoparticles.

(e) In the gel denoted as (c) or (d), the starch-coated IONPs may be selected from the group consisting of 20 nm nanomag-D spioplain IONPs, 80 nm BNF Dextran IONPs and 100 nm nanomag-D spioplain IONPs.

(f) In the gel denoted as (c) or (e), the polysaccharide and PBS may be mixed at a ratio of 3 g polysaccharide to 7 mL PBS.

(g) In the gel denoted as (c) or (e), the polysaccharide and PBS may be mixed at a ratio of 1 g polysaccharide to 9 mL PBS.

(h) In the gel denoted as (a)-(g), the gel may include a protein or protein precursor.

(i) In the gel denoted as (h), the protein or protein precursor may be fibrinogen, collagen, fibrin, or combinations thereof.

(j) In the gel denoted as (e), The gel of claim 4, the 20 nm nanomag-D spioplain IONPs may be at a concentration of 108 mg/mL, the 80 nm BNF Dextran IONPs may be at a concentration of 38 mg/mL and the 100 nm nanomag-D spioplain IONPs may be at a concentration of 10 mg/mL.

(k) In the gel denoted as (b)-(j), the polysaccharide derivatives may include carboxymethylcellulose and agar.

(l) In the gel denoted as (b)-(j), the polysaccharide derivative may be carboxymethylcellulose, the iron oxide nanoparticles may have a biocompatible coating, and the gel may further include fibrinogen.

(m) In the gel denoted as (b)-(j), the polysaccharide derivative may be agar, the iron oxide nanoparticles may have a biocompatible coating, and further the gel may further include fibrinogen.

(n) In the gel denoted as (a)-(m), the polysaccharide may be a retrograded polysaccharide.

(o) In the gel denoted as (a)-(m), the gel may be pre-heated to retrograde the polysaccharide and increase viscosity of the gel.

(p) In the gel denoted as (a)-(o), the polysaccharide may be a polysaccharide that is subjectable to retrogradation.

(q) In the gel denoted as (a)-(p), a biocompatible antibody target that is recognizable to cancer cells may be coated onto or otherwise incorporated with the IONPs, to enhance uptake of the IONPs by cancer cells.

(r) A method of targeted hyperthermia treatment following resection includes applying a biodegradable iron oxide nanoparticle gel to a tumor bed following resection,

allowing the gel to remain on the tumor bed for an elution period, and heating the nanoparticle gel using an external AC magnetic field. The gel is allowed to drain from the tumor bed for a drainage period, and the external AC magnetic field is applied proximate a lymph node or nodes within the lymphatic drainage of the tumor bed, to heat nanoparticles metastasized from the tumor bed to the lymph node or nodes and ablate cancer cells within the lymph node or nodes.

(s) In the method denoted as (r), the elution period may be 30 minutes to four hours.

(t) In the methods denoted as (r) or (s), the drainage period may be one to 24 hours.

(u) In the methods denoted as (r)-(t), the gel may be injected over the tumor bed or coating the gel over the tumor bed.

(v) In the methods denoted as (r)-(u), the gel may be laparoscopically applied to the tumor bed.

(w) In the methods denoted as (r)-(v), the gel may be a polysaccharide-based gel.

(x) In the methods denoted as (r)-(v), the gel may be a protein-based or protein precursor-based gel.

(y) In the methods denoted as (r)-(x), the gel may be a mixture of a polysaccharide, phosphate buffered saline and starch-coated iron oxide nanoparticles.

(z) In the methods denoted as (r)-(y), the gel may be a mixture of at least one polysaccharide, phosphate buffered saline, a protein or protein precursor and starch-coated iron oxide nanoparticles.

(aa) In the methods denoted as (r)-(z), the nanoparticle gel may include nanoparticles coated or otherwise associated with a tumor-specific antibody target.

(bb) A diagnostic method for locating and mapping cancerous cells within a patient's body includes applying an iron oxide nanoparticle gel to a tumor bed, following resection, allowing the gel to sit for an elution period and allowing the gel to drain for a drainage period. One or more imaging studies are performed to image iron oxide nanoparticles eluted from the gel and taken up by cancerous cells within the body; and imaging is repeated until mapping of cancerous cells within the body is complete.

(cc) In the method denoted as (bb), the imaging studies may be timed from a time of application of the iron oxide nanoparticle gel.

(dd) In the methods denoted as (bb) and (cc), an alternating magnetic field may be induced to heat the nanoparticles at the tumor bed, following the elution period.

(ee) In the methods denoted as (bb)-(cc), the one or more imaging studies may be performed during the elution period.

(ff) A biodegradable iron oxide nanoparticle gel includes a carrier matrix including fibrin glue, and biocompatibly-coated iron oxide nanoparticles. The gel has sufficient deformability to integrate into a diffuse tumor site; the gel adheres to tissue and has sufficient mechanical properties to remain in place; the gel is capable of releasing the nanoparticles for uptake by cancerous cells proximate the tumor bed, and the gel maintains the mechanical properties at temperatures ranging from room temperature to a hyperthermia range of 43-37° C. for at least a therapeutic time period.

(gg) In the method denoted as (ff), the gel may include a polysaccharide.

(hh) In the method denoted as (gg), the polysaccharide may be a retrograded polysaccharide.

(ii) A biodegradable iron oxide nanoparticle gel includes a carrier matrix including collagen, and biocompatibly-coated iron oxide nanoparticles. The gel has sufficient deformability to integrate into a diffuse tumor site; the gel adheres to tissue and has sufficient mechanical properties to remain in place; the gel is capable of releasing the nanoparticles for uptake by cancerous cells proximate the tumor bed, and the gel maintains the mechanical properties at temperatures ranging from room temperature to a hyperthermia range of 43-37° C. for at least a therapeutic time period.

(jj) In the method denoted as (ii), the carrier matrix may include thrombin.

(kk) In the methods denoted as (ii) and (jj), the biocompatibly-coated iron oxide nanoparticles may be starch-coated nanoparticles.

(ll) In the methods denoted as (bb)-(kk), the carrier matrix may further include a polysaccharide.

(mm) In the method denoted as (11), wherein the polysaccharide may be a retrograded polysaccharide.

What is claimed is:

1. A biodegradable iron oxide nanoparticle gel, comprising
at least one polysaccharide; and
iron oxide nanoparticles;
wherein the gel has sufficient deformability to integrate into a diffuse tumor site;
wherein the gel adheres to tissue and has sufficient mechanical properties to remain in place;
wherein the gel releases the iron oxide nanoparticles for uptake by cancerous cells at the tumor site; and
wherein the gel maintains the mechanical properties at temperatures ranging from room temperature to a hyperthermia range of 43-37° C.

2. The gel of claim 1, the polysaccharide being selected from the group consisting essentially of pullulan, cellulose, amylose, amylopectin, agarose, glycogen, chitin, callose, laminarin, chrysolaminarin, xylan, a hemicellulose such as arabinoxylan, mannan, fucoidan, galactomannan and derivatives thereof, alone or in combination

3. The gel of claim 1, the gel comprising a mixture of the at least one polysaccharide, phosphate buffered saline (“PBS”) and starch-coated iron oxide nanoparticles (“IONPs”).

4. The gel of claim 3, the starch-coated IONPs selected from the group consisting of 20 nm nanomag-D spioplain IONPs, 80 nm BNF Dextran IONPs and 100 nm nanomag-D spioplain IONPs.

5. The gel of claim 1, further comprising a protein or protein precursor selected from the group of fibrin, fibrinogen, collagen and combinations thereof.

6. The gel of claim 4, the 20 nm nanomag-D spioplain IONPs being at a concentration of 108 mg/mL, the 80 nm BNF Dextran IONPs being at a concentration of 38 mg/mL and the 100 nm nanomag-D spioplain IONPs being at a concentration of 10 mg/mL.

7. The gel of claim 2, the polysaccharide derivatives including carboxymethylcellulose and agar.

8. The gel of claim 7, the iron oxide nanoparticles comprising iron oxide nanoparticles having a biocompatible coating, and further comprising fibrinogen.

9. The gel of claim 1, wherein the polysaccharide is a retrograded polysaccharide.

10. The gel of claim 1, further comprising a biocompatible antibody target recognizable to cancer cells, coated onto or otherwise incorporated with the IONPs, to enhance uptake of the IONPs by cancer cells.

11. A method of targeted hyperthermia treatment following resection, comprising:
applying a biodegradable iron oxide nanoparticle gel to a tumor bed following resection;
allowing the gel to remain on the tumor bed for an elution period;
heating the nanoparticle gel using an external AC magnetic field;
allowing the gel to drain from the tumor bed for a drainage period; and
applying the external AC magnetic field proximate a lymph node or nodes within the lymphatic drainage of the tumor bed, to heat nanoparticles metastasized from the tumor bed to the lymph node or nodes and ablate cancer cells within the lymph node or nodes.

12. The method of claim 11, wherein the gel is a polysaccharide-based gel.

13. The method of claim 11, wherein the gel is a protein-based or protein precursor-based gel.

14. The method of claim 11, wherein the gel comprises a mixture of a polysaccharide, phosphate buffered saline and starch-coated iron oxide nanoparticles.

15. The method of claim 14, wherein the gel further comprises a protein or protein precursor.

16. The method of claim 11, the nanoparticle gel including nanoparticles coated or otherwise associated with a tumor-specific antibody target.

17. A diagnostic method for locating and mapping cancerous cells within a patient's body, comprising:
applying an iron oxide nanoparticle gel to a tumor bed, following resection;
allowing the gel to sit for an elution period;
allowing the gel to drain for a drainage period;
performing one or more imaging studies to image iron oxide nanoparticles eluted from the gel and taken up by cancerous cells within the body; and
repeating the step of imaging until mapping of cancerous cells within the body is complete.

18. The method of claim 17, further comprising the step of timing the imaging studies from a time of application of the iron oxide nanoparticle gel.

19. The method of claim 17, further comprising the step of inducing an alternating magnetic field to heat the nanoparticles at the tumor bed, following the elution period.

20. The method of claim 27, wherein the one or more imaging studies are performed during the elution period.

21. A biodegradable iron oxide nanoparticle gel, comprising
a carrier matrix including fibrin glue; and
biocompatibly-coated iron oxide nanoparticles;
wherein the gel has sufficient deformability to integrate into a diffuse tumor site;
wherein the gel adheres to tissue and has sufficient mechanical properties to remain in place;
wherein the gel releases the iron oxide nanoparticles for uptake by cancerous cells at the tumor site; and
wherein the gel maintains the mechanical properties at temperatures ranging from room temperature to a hyperthermia range of 43-37° C.
